(19) United States
(12) Patent Application Publication
(10) Pub. No.: US 2025/0332398 A1
(43) Pub. Date: Oct. 30, 2025

(54) SYSTEMS AND METHODS FOR DELIVERING A FLUID FROM A NON-STERILE FIELD TO A STERILE FIELD

(71) Applicant: Ann and Robert H. Lurie Children's Hospital of Chicago, Chicago, IL (US)

(72) Inventors: Seth D. Goldstein, Chicago, IL (US); Jennifer Lynn Fortson, Homewood, IL (US)

(21) Appl. No.: 18/864,221

(22) PCT Filed: May 9, 2023
(86) PCT No.: PCT/US2023/066765

§ 371 Date: Nov. 8, 2024

Related U.S. Application Data
(60) Provisional application No. 63/339,777, filed on May 9, 2022.

(51) Int. Cl. A61M 39/18

(52) U.S. Cl. CPC A61M 39/18

(57) ABSTRACT

A fluid passthrough device delivers a fluid from a non-sterile field to a sterile field in an operating room. The fluid passthrough device includes a housing extending from a first surface facing the sterile field to a second surface facing the non-sterile field. A receiving interface is formed in the second surface of the housing to receive a fluid reservoir containing a fluid, and a dispensing interface is formed in the first surface of the housing to receive the fluid from the receiving interface and dispense the fluid into the sterile field. The fluid passthrough allows for fluid to be delivered in a sterile manner from the non-sterile field directly to the sterile field, improving the overall surgical workflow and minimizing the number of transfers of sterile fluid.

### CROSS-REFERENCE TO RELATED APPLICATIONS

This application claims the benefit of U.S. Provisional Patent Application Ser. No. 63/339,777, filed on May 9, 2022, and entitled “ATOMIZED ANTISEPTIC ADMINISTRATION DURING SURGICAL LAPAROSCOPY,” which is herein incorporated by reference in its entirety.

### BACKGROUND

Over 20 million invasive surgeries are performed in the U.S. annually. Each invasive procedure requires an established sterile field within which objects and operating room personnel must be scrubbed and sterile. Once the sterile field is established, it is best practice to maintain the sterile field by ensuring no unsterile objects or people enter the sterile field. It is also considered best practice to continuously face an inside of the sterile field, meaning the operating room personnel should not turn to look for objects on tables, turn to receive tools and fluids, or move around the sterile field. In some instances, operating room personnel may be forced to make the decision between leaving the sterile field with subsequent re-entry, or receiving supplies from outside of the sterile field if key materials, such as liquid medications and other fluids, that are needed for the patient are depleted or absent.

As the sterile field may be a small area, reducing the number of operating room personnel within the sterile field is ideal to reduce overcrowding the surgeon(s), while also reducing the risk of contamination. The potential for staff outside of the sterile field to sterilely assist in the surgical procedures may reduce the overcrowding of physicians, while reducing the likelihood of infection post procedure. The interface between sterile and unsterile personnel must be carefully choreographed to pass supplies into the sterile field. For instance, some items are separately “peel-packaged,” for facile opening. However, the transfer of fluids-including saline, antiseptics, and liquid medications-poses a unique challenge. These materials are typically sterile inside their packaging, but are not additionally autoclaved due to expense or heat-sensitivity. Accordingly, the transfer of these fluids is unwieldy, at risk of inadvertent contamination, and can expose personnel to needlestick injury. Thus, there is a need for a simple and time-efficient device that provides non-sterile staff further opportunity to transfer fluid materials to sterile staff during surgical procedures.

### SUMMARY OF THE DISCLOSURE

The present disclosure addresses the aforementioned drawbacks by providing a fluid passthrough device for delivering fluid from a non-sterile field to a sterile field. One or more fluid reservoirs are coupled to a first side of the device. The device includes one or more channels which allow the fluid to flow from the first side of the device to a second side of the device. The fluid is then capable of being delivered to the sterile field via a tube, a spout, a syringe, an atomizer, or any other operating room delivery mechanism.

It is one aspect of the present disclosure to provide a fluid passthrough device to deliver a fluid from a non-sterile field to a sterile field in an operating room. The fluid passthrough device includes a housing extending from a first surface facing the sterile field to a second surface facing the non-sterile field. A receiving interface is formed in the second surface of the housing to receive a fluid reservoir containing a fluid, and a dispensing interface is formed in the first surface of the housing to receive the fluid from the receiving interface and dispense the fluid into the sterile field.

It is another aspect of the present disclosure to provide a method of sterilely transporting a fluid from a non-sterile field to a sterile field using a fluid passthrough device having a housing extending from a first surface facing the sterile field to a second surface facing the non-sterile field, a receiving interface formed in the second surface of the housing, a dispensing interface formed in the first surface of the housing, a channel fluidically coupling the receiving interface to the dispensing interface, and a valve controlling fluid flow through the channel. The method includes receiving a reservoir containing a fluid with the receiving interface of the fluid passthrough device, such that the reservoir is in fluid communication with the channel and such that the fluid remains sterile. The valve is then actuated to an open position, transporting the fluid from the fluid reservoir through the channel to the dispensing interface. The fluid is then dispensed from the dispensing interface to a target area in the sterile field.

The foregoing and other aspects and advantages of the present disclosure will appear from the following description. In the description, reference is made to the accompanying drawings that form a part hereof, and in which there is shown by way of illustration one or more embodiments. These embodiments do not necessarily represent the full scope of the invention, however, and reference is therefore made to the claims and herein for interpreting the scope of the invention.

### BRIEF DESCRIPTION OF THE DRAWINGS

FIG. 1 is a side view of an example fluid passthrough device, in accordance with the disclosed subject matter.

FIG. 2 illustrates the fluid passthrough device of FIG. 1 disposed on a cart.

FIG. 3 illustrates an example of a location of the fluid passthrough device of FIG. 1 within an operating room.

FIG. 4 illustrates an example section of the fluid passthrough device FIG. 1.

FIG. 5 illustrates an example section of the fluid passthrough device of FIG. 1 in fluid communication with a pump.

FIG. 6 illustrates an example section of the fluid passthrough device FIG. 1 coupled to a fluid reservoir.

FIG. 7 illustrates an example section of the fluid passthrough device FIG. 1 with a syringe inserted through the section.

FIG. 8 illustrates an example section of the fluid passthrough device FIG. 1 including an electronic actuator.

FIG. 9 is a method for using the fluid passthrough device, in accordance with an example of the disclosed subject matter.

FIG. 10 is another method for utilizing a fluid passthrough device, in accordance with an example of the disclosed subject matter.

FIG. 11 illustrates an example laparoscopic system for delivering antiseptic solution. The system allows for a sterile/non-sterile duality once the tubing for the loading mechanism has been passed off the sterile field into the container of antiseptic solution. The blown-up portion illustrates that there are two apertures (e.g., an inlet and an outlet) into the portion of the device with a reservoir.

FIG. 12 illustrates an apparatus to close off the nozzle lumen (i.e., outlet) when negative pressure is pulling fluid into the reservoir via the inlet.

FIG. 13 illustrates an apparatus to open the nozzle lumen (i.e., outlet) and to close the inlet while also detaching the tubing from the inlet such that the tubing falls off outside of the sterile field. Generation of positive pressure in the reservoir can eject fluid through the nozzle.

### DETAILED DESCRIPTION

In general, the systems and methods described in the present disclosure implement the following functions: loading a fluid into a reservoir, maintaining a sterile field during loading, transferring, and dispensing of the fluid out of the reservoir to the sterile field.

Described here are systems and methods for sterilely delivering sterile fluid from a non-sterile field to the sterile field, thereby reducing the risk of contamination, risk of needlestick injury, risk of medication error/mislabel, and need for simultaneous attention of personnel both in and out of the sterile field, while reducing the risk of contamination, risk of needlestick injury, risk of medication error/mislabel, and need for simultaneous attention of personnel both in and out of the sterile field. For instance, the disclosed systems and methods minimize the number of transfers of fluid reservoirs across the sterile field in the operating room (“OR”) preparation, as well as during the procedure. As a result of these and other features and advantages, the disclosed systems and methods aim to reduce infection rates from invasive surgeries and improve surgical workflow in the operating room.

The disclosed systems and methods provide a single interface that can be adapted for multiple different types of fluid to be delivered to the sterile field at various rates and in multiple forms, with the help of non-sterile staff. The disclosed system potentially allows sterile and/or non-sterile staff to control at least fluid delivery of a liquid to the sterile field. The fluid can be transferred from outside the sterile field to within the sterile field while maintaining the sterile nature of both the fluid and sterile field, thereby allowing for simpler preparation during an operating room procedure.

As a non-limiting example, fluid passthrough device may be used in an operating room to help deliver fluid from a non-sterile field to a sterile field. In some embodiments, the fluid passthrough device includes a housing extending from a first surface to a second surface, where the first surface faces the sterile field and the second surface faces the non-sterile field. The housing may be a solid body, or may be hollow. The second surface may have one or more receiving interfaces for receiving a container or other reservoir of fluid from the non-sterile field, and the first surface may have one or more dispensing interfaces for dispensing the fluid received by the container(s) operatively engaging with the interface(s) of the second surface of the fluid passthrough device.

In some embodiments, the fluid passthrough device includes one or more channels extending through the housing (i.e., continuously through the housing) to fluidically couple the receiving interface(s) with the dispensing interface(s), so that fluid from the fluid reservoirs may flow through the fluid passthrough device. The fluid passthrough device may be coupled to a fluid transport mechanism or delivery mechanism, configured to aid the transfer of the fluid from the fluid passthrough device to the target area in the sterile field.

In some embodiments, the fluid passthrough device includes one or more valves to regulate the flow of fluid through each of the channels. In some embodiments, each of the valves include an actuator that interface with the operating room personnel to operate the flow rate and volume of the fluid delivered to the target area. The one or more valves may be incorporated as part of the dispensing interface(s), or may be separate components from the dispensing interface(s).

In some embodiments, the fluid passthrough device is configured to transfer multiple types of fluid, e.g., gas, liquid, gel, etc. As used herein “fluid” or “fluids” is defined as any liquid, plasma, gas, gel, foam, paste, or other substance that can deform, for example, under external pressure or other force and tend to flow or conform to the outline of its container. In some embodiments, the fluid passthrough device is modular, and may include multiple sections. In some embodiments, each section is configured to transfer a single fluid type. The sections may be added or removed from the fluid passthrough device to save space or to accommodate the transfer of more fluids. In these instances, each section of the fluid passthrough device may include a housing extending from a first surface that faces the sterile field and a second surface that faces the non-sterile field, with one or more channels fluidically coupling a receiving interface on the second surface with a dispensing interface on the first surface.

In some embodiments, the fluid passthrough device includes a pump or is fluidly connected to a pump to aid in the transfer of the fluid from the fluid reservoir to the target area in the sterile field.

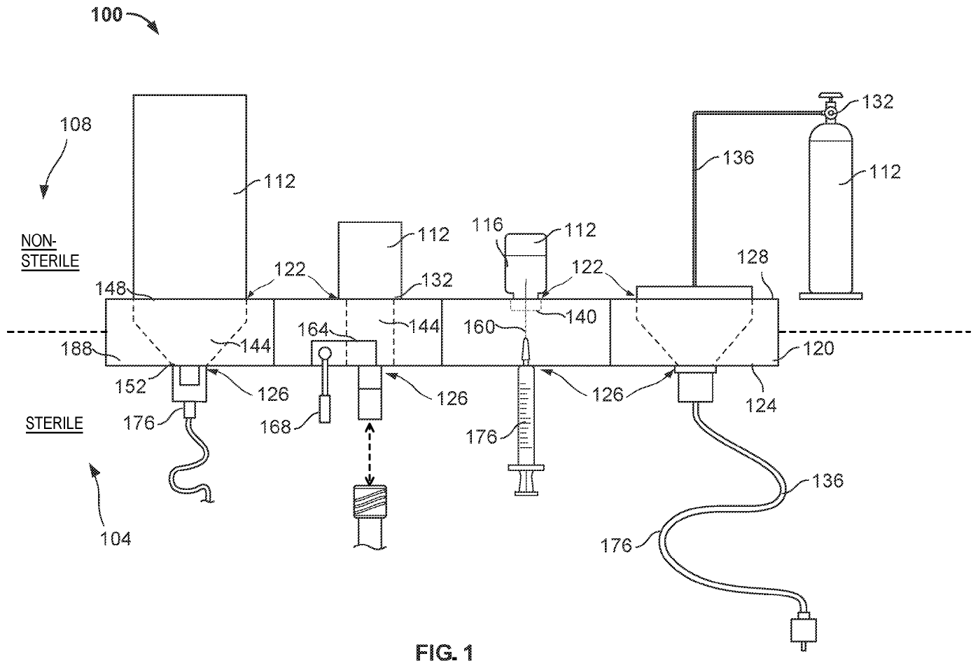

Referring to FIG. 1, a fluid passthrough device 100 is illustrated spanning a sterile field 104 and a non-sterile field 108. The fluid passthrough device 100 is capable of receiving one or more fluid reservoirs 112, and transferring a fluid 116 within the one or more fluid reservoirs 112 from the non-sterile field 108 to the sterile field 104. In some embodiments, the fluid passthrough device 100 may include a housing 120 extending from a first surface 124 to a second surface 128, where the first surface 124 faces the sterile field 104 and the second surface 128 faces the non-sterile field 108.

The second surface 128 has formed therein one or more receiving interfaces 122 for receiving fluid 116 from a fluid reservoir 112. As will be described below in more detail, the receiving interface 122 may operatively engage a fluid reservoir 112, may otherwise be fluidically coupled to a fluid reservoir 112, or may otherwise receive fluid 116 from the fluid reservoir 112. The receiving interface 122 may include a recessed portion formed in the second surface 128 of the housing 120, an aperture formed in the second surface 128 of the housing 120, or the like. In some examples, the receiving interface 122 may be threaded, may be sized to provide an interference fit, or the like.

Likewise, the first surface 124 has formed therein one or more dispensing interfaces 126 for dispensing a fluid 116 received from a fluid reservoir 112 by a corresponding receiving interface 122. As will be described below in more detail, the dispending interfaces 126 may include mechanisms for dispensing a fluid 116, such as apertures, spouts, valves, pneumatic dispensers, non-contact dispensers (e.g., motion activated dispensers), peristaltic pump dispensers, rotary pump dispensers, metering pump dispensers, spraying dispensers, tubing dispensers, fluid couplings, or the like.

In some embodiments, the housing 120 may be a single body. In other examples, the housing 120 may include two or more stacked components that may be interchangeable. For instance, the housing 120 may include a first piece that may be stacked on top of a second piece. In some embodiments, the housing 120 may be a solid body that is integrally formed. In some other embodiments, the housing 120 may be a hollow body that is integrally formed. As noted above, in other examples, the housing 120 may include two or more stacked components that are coupled together (e.g., operatively engaged with one another). In these instances, the different components of the housing 120 that are separable may be coupled by any suitable means (e.g., a latch, a fastener, press-fit, snap-fit, threading, overmolding, etc.). In general, the housing 120 may be any 3-D shape (e.g., rectangular, spherical, trapezoidal, cylindrical, etc.).

The first surface 124 is sterile and considered to be within the sterile field 104, to ensure the fluid 116 delivered to the sterile field 104 from the one or more fluid reservoirs 112 remains sterile. In some embodiments, the second surface 128 may be considered to be in the sterile field 104, even though it faces the non-sterile field. In these instances, a fluid reservoir 112 may interface with the receiving interface(s) 122 via a sterile coupling in the non-sterile field. In some embodiments, the fluid passthrough device 100 is not in the sterile field 104. In these instances, the dispensing interface(s) 126 may provide a sterile fluid connection to within the sterile field, or the housing 120 of the fluid passthrough device 100 may be located outside of, but at the edge of, the sterile field, such that the first surface 124 and dispensing interface(s) 126 are contained within the sterile field. In some embodiments, the fluid passthrough device 100 is manufactured from an autoclavable material, allowing the fluid passthrough device 100 to be completely sterilized before each use. In some embodiments, the fluid passthrough device 100 is a single use tool. When the housing 120 of the fluid passthrough device 100 is composed of stacked components, each component may be provided as a separate component that can be interchangeably used to construct a fluid passthrough device 100. Similarly, when the fluid passthrough device 100 includes modular sections, each section may be provided as a separate component that can be interchangeably used to construct a fluid passthrough device 100. In some embodiments, one or more of the stacked components, the modular sections, and/or the fluid reservoirs 112 may be single use components.

Still referring to FIG. 1, the one or more fluid reservoirs 112 may be suited to house and deliver any type of the fluid 116 (e.g., drugs, medicine, antiseptic, gas, gels, blood, water, saline, etc.). Suitable fluid reservoirs 112 may include gas cannisters, fluid bags, bottles, syringes, glass vials, plastic vials, reservoirs attached to a pump or atomizer, a funnel, etc. Each fluid reservoir 112 may include a reservoir opening 132 that is coupled to the receiving interface 122 of the fluid passthrough device 100. In some embodiments, each of the reservoir openings 132 is connected to the receiving interface 122 of the fluid passthrough device 100 via a tube 136. The reservoir openings 132, or the tubes 136, may provide fluid communication between the fluid reservoirs 112 and the fluid passthrough device 100 via the receiving interfaces 122. The reservoir openings 132 may be sterilized to ensure that the fluid 116 within each of the one or more fluid reservoirs 112 remains sterile while the fluid 116 is delivered from the non-sterile field 108 to the sterile field 104. In some embodiments, one or more of the reservoir openings 132 may include a lid or seal 140. The seals 140 may be configured to release the fluids 116 within the fluid reservoirs 112 when the seals 140 are punctured or otherwise actuated.

In some embodiments, the one or more fluid reservoirs 112 may be received by the receiving interface(s) 122 on the second surface 128 of the housing 120. The fluid 116 from the one or more fluid reservoirs 112 may flow through one or more channels 144 that extend through the housing to fluidically couple the receiving interface(s) 122 to the dispensing interface(s) 126, effectively transferring fluid from the non-sterile field 108 to the sterile field 104. The one or more channels 144 may be separately configured to retain and transport a different type of the fluid 116. In some embodiments, the one or more channels 144 each include a first opening 148 fluidically coupled to the receiving interface 122, and a second opening 152 fluidically coupled to a dispensing interface 126. In some embodiments, the fluids 116 from the one or more fluid reservoirs 112 may flow through the first openings 148 to the one or more channels 144 out the second openings 152.

In some embodiments, the fluid reservoir 112 may be decoupled from the receiving interface 122 during a procedure. The fluid reservoir 112 may be decoupled because it is depleted, or because a different fluid is needed. Once the fluid reservoir 112 is decoupled, a second fluid reservoir may be coupled to the receiving interface 122 to continue to provide fluid for the procedure.

In some embodiments, each of the fluid reservoirs 112 is in fluid communication with only one of the one or more channels 144. In some embodiments, each of the fluid reservoirs 112 is in fluid communication with more than one of the one or more channels 144. In some embodiments, more than one of the fluid reservoirs 112 may be in fluid communication with one of the one or more channels 144 to allow for mixing of the fluid 116 within the fluid passthrough device 100.

Still referring to FIG. 1, as discussed above the fluid 116 from each fluid reservoir 112 may flow through the fluid passthrough device 100 via the one or more channels 144. In some embodiments, the one or more channels 144 may taper. A diameter of one or more of the channels 144 may increase or decrease between the first opening 148 and the second opening 152. In some embodiments, one or more of the channels 144 may be straight. In some embodiments, one or more of the channels 144 may be arcuate or include one or more bends. In some examples, the housing 120 is a solid body and the one or more channels 144 are formed in the body of the housing 120. In some other examples, the housing 120 is a hollow body and the one or more channels 144 may include tubing that fluidically couples the receiving interfaces 122 to the dispensing interfaces 126.

Still referring to FIG. 1, as described above, each of the fluid reservoirs 112 may be coupled to the fluid passthrough device 100 via a receiving interface 122. The receiving interface 122 may include a coupling that may be leak proof, ensuring the efficient transfer of any type of liquid, gas, gel, or other type of the fluid 116 from the fluid reservoir 112 to the sterile field 104. In these instances, the couplings used for the receiving interfaces 122 may be any suitable medical device coupling (e.g., threading, press fit/friction fit, Luer taper, Tuohy Borst adapter, a needle, Chemetron Quick Connects, or other suitable coupling means). In some embodiments, one or more of the receiving interfaces 122 is associated with each of the one or more channels 144.

As described above, in some embodiments, the receiving interfaces 122 are disposed on the first openings 148 of the channels 144. In some embodiments, the receiving interfaces 122 may be disposed on the second surface 128. In some embodiments, the receiving interfaces 122 may be recessed within the housing 120 (e.g., recessed from the second surface 128). In some embodiments, one of the receiving interfaces 122 may be integral to one of the channels 144 and may hold one of the fluid reservoirs 112, such that a portion of the fluid reservoir 112 extends into the sterile field 104. In some embodiments, one of the receiving interfaces 122 may be a funnel, which allows personnel to pour the fluid 116 from the one of fluid reservoirs 112 into the fluid passthrough device 100. In some embodiments, one or more of the receiving interfaces 122 may be integral to the housing 120. In some embodiments, the receiving interfaces 122 may be separable from the fluid passthrough device 100. In some embodiments, the receiving interfaces 122 are sterile. In some other embodiments, the receiving interfaces 122 may not be sterile.

The one or more channels 144 may each include a cannula 160 through which the fluid 116 flows. In some embodiments, only some of the one or more channels 144 include the cannula 160. In some embodiments, one or more of the cannulas 160 may be a tube. In some embodiments, one or more of the cannulas 160 may be a needle. Similar to the one or more channels 144, a diameter of the cannulas 160 may also vary between the first opening 148 and the second opening 152. In some embodiments, the cannulas 160 may be removable from the channels 144 to allow for simpler sterilization. In some embodiments, the cannulas 160 may be coupled to the fluid reservoirs 112, in order to provide fluid communication between the first openings 148 and the second openings 152. In some embodiments, the cannulas 160 may be inserted through the first openings and through the second openings 152 to access the fluid reservoirs 112.

In some embodiments, fluid flow through each of the one or more channels 144 or to the one or more channels 144 may be restricted by one or more valves 164. In some embodiments, fluid flow through only some of the one or more channels 144 may be restricted by the valves 164. In some embodiments, the one or more valves 164 may be actuatable between a closed position, one or more partially open positions, and a totally open position. The one or more valves 164 may restrict fluid flow from the non-sterile field 108 to the sterile field 104, from the sterile field 104 to the non-sterile field 108, or restrict flow to both the sterile field 104 and the non-sterile field 108.

In some embodiments, the one or more valves 164 may be disposed on the reservoir openings 132 of the fluid reservoirs 112. In some embodiments, the one or more valves 164 may be coupled to or integral with the dispensing interfaces 126, coupled to or integral with the receiving interfaces 122, or otherwise disposed anywhere along the one or more channels 144. In some embodiments, the one or more valves 164 may be coupled to the fluid passthrough device 100 or the reservoir openings 132 via a threading, friction fit, over molding, adhesive, Tuohy Borst adapter, Luer taper, or other suitable coupling means. In some embodiments, the one or more valves 164 may be integral to the housing 120 of the fluid passthrough device 100 or the fluid reservoir 112. In some embodiments, one or more of the valves 164 may be removable from the fluid passthrough device 100 or fluid reservoir 112. The one or more valves 164 may be any type of valve 164 that regulates fluid flow. In some embodiments, the one or more valves 164 may include different valve types, as described below.

In some embodiments, one or more of the valves 164 may be a check valve or a one-way valve, configured to either restrict fluid flow within the fluid passthrough device 100 in a sterile to non-sterile direction, or in a non-sterile to sterile direction. In some embodiments, one or more of the valves 164 may be a relief valve, configured to allow fluid to flow within the fluid passthrough device 100 either in a sterile to non-sterile direction, or in a non-sterile to sterile direction, when pressure within the fluid reservoir 112 exceeds a set pressure. In some embodiments, one or more of the valves 164 may be an on-off valve configured to allow fluid to flow within the fluid passthrough device 100 either in a sterile to non-sterile direction, or in a non-sterile to sterile direction when actuated to an open position.

Each of the one or more valves 164 that is the on-off valve may be actuated between the open position and a closed position via an actuator 168. In some embodiments, each of the actuators 168 may only partially open one of the valves 164. In some embodiments, each of the actuators 168 may actuate more than one of the valves 164. Each of the actuators 168 may be disposed anywhere along the fluid circuit (i.e., on the fluid reservoirs 112, the receiving interfaces 122, the tubes 136, the channels 144, the dispensing interfaces 126, or a fluid dispenser 176 described below). In some embodiments, the actuators 168 may be disposed in the sterile field 104. In some embodiments, the actuators 168 may be disposed in the non-sterile field 108.

In some embodiments, one or more of the actuators 168 may include a knob or rotatable component. In some embodiments one or more of the actuators 168 may be a lever or switch that is flipped or otherwise turned between the open position and the closed position. In some embodiments, one or more of the actuators 168 may include a component that is translated along the fluid reservoir 112 or along the fluid passthrough device 100, in a sterile to non-sterile direction, or in a non-sterile to sterile direction. In some embodiments, one or more of the actuators 168 may be a button that is pressed to open or close one or more of the valves 164. In some embodiments, one or more of the actuators 168 may actuate one or more of the valves 164 electrically between the open position and the closed position when an electronic signal is received from a remote, or processor. The electronic signal may be created by the push of a button on the remote or other user interface such as a computer or tablet. In some embodiments, one or more of the valves 164 may be actuated to the open/closed position when the processor generates the electronic signal in response to a sound (e.g., voice activation) or from a gesture or movement by the operators. For instance, in some examples a valve 164 may be actuated to an open position by detecting motion (e.g., a user waving or otherwise passing their hand by a motion sensor coupled to an actuator 168). In some embodiments, the processor may display a graphical user interface on a monitor or screen to allow the non-sterile operators or sterile operators to interact with the electrically activated valve. In some embodiments, the operators may interact with the processor via touch screen, a mouse, a joystick, a keyboard, or other manipulatable controllers.

Still referring to FIG. 1, fluid 116 may flow through the fluid passthrough device 100 via the one or more channels 144 (or cannulas 160) toward the sterile field 104. The fluid 116 may leave each of the one or more channels 144 via a dispensing interface 126. The dispensing interfaces 126 may include couplings that may be leak proof, ensuring the efficient transfer of any type of liquid, gas, gel, or other flowing substance from the fluid passthrough device 100 to the sterile field 104. The dispensing interface 126 may include any suitable medical device coupling (e.g., threading, press fit/friction fit, Luer taper, Tuohy Borst adapter, a needle, Chemetron Quick Connects, or other suitable coupling means). In some embodiments, dispensing interface 126 coupled to each of the one or more channels 144 may be disposed on first surface 124 of the housing 120. In some embodiments, the dispensing interface 126 may be recessed within the housing 120 (e.g., recessed from the first surface 124).

In some embodiments, each of the dispensing interfaces 126 is coupled to one of the fluid dispensers 176. The fluid dispensers 176 may be suited to house and deliver any type of fluid 116 (e.g., drugs, antiseptic, gas, gels, blood, water, saline, mist, etc.). Suitable fluid dispensers 176 may include one or a combination of spouts, gas cannisters, fluid bags, syringes, glass vials, plastic vials, reservoirs, spray nozzles, pumps, tubes, atomizers, other dispensing mechanisms described above, etc. The fluid dispensers 176 may include a dispense mechanism. The dispense mechanism may include a plunger (e.g., like a syringe), a spring, a trigger spray mechanism, a valve, an atomizer nozzle, an injection needle for intramuscular/subcutaneous injections, a nebulizer, or other dispensation mechanism for dispensing the fluid 116 to a target area 180. In some embodiments, the fluid dispenser 176 may interface with an exterior fluid dispense mechanism such as a syringe, that draws fluid from the fluid dispenser 176.

In some embodiments, the fluid dispenser 176 may be detachable from the passthrough device 100 in order to allow the operators for increased freedom of movement. In some embodiments, the fluid dispenser 176 may be connected to the passthrough device 100 via the one or more tubes 136. In some embodiments, the tube 136 can be detached from the passthrough device 100 or the fluid dispenser 176. In some embodiments, the tube 136 may be connected using a friction fit to one of the receiving interfaces 122 or dispensing interfaces 126 of the fluid passthrough device 100. In some embodiments, a mechanical ejection of the tube 136 can be used to detach the tubing from the fluid dispenser 176 or the fluid passthrough device 100. In some embodiments, a quick disconnect coupling can be used to couple the tube 136 to the receiving interface 122 or the fluid dispenser 176. In some embodiments, a threaded nut can be used, such that unthreading the nut can detach the tube 136 from the receiving interface 122 or the fluid dispenser 176. In still other embodiments, a prepositioned hand-squeezed crimp and/or cut in the tube 136 can be used to provide detachment of the tube from the passthrough device 100. For instance, having a prepositioned crimp and/or cut in the tube 136 can enable quick detachment of the tube 136 along the crimp and/or cut after loading of the fluid dispenser 176.

Still referring to FIG. 1, the fluids 116 may be biased toward the sterile field 104 using any suitable mechanism. In some embodiments, the fluids 116 may biased toward the sterile field 104 via gravity, by supporting one or more of the fluid reservoirs 112 and the fluid passthrough device 100 above an exit point of one or more of the fluid dispensers 176. In some embodiments, the fluids 116 may be biased toward the sterile field 104 using a pump 184, anywhere along the fluid circuit. In some embodiments, the pump 184 may be disposed within the housing 120 of the fluid passthrough device 100. In some embodiments, the fluids 116 may be biased toward the sterile field 104 by applying a mechanical pressure to one or more of the fluid reservoirs 112 via one or more plungers or by squeezing one or more sides of the fluid reservoirs 112.

Still referring to FIG. 1, in some embodiments the fluid passthrough device 100 may be composed of one or more sections 188. The fluid passthrough device 100 may include any number of the one or more sections 188. The one or more sections 188 may each include a housing 120 extending from a first surface 124 to a second surface 128, a receiving interface 122, a dispensing interface 126, a channel 144, a valve 164, and an actuator 168. In some embodiments, each of the one or more sections 188 may be configured to transfer a certain type of the fluid 116 to the sterile field 104. Each of the one or more sections 188 may be configured to accept one or more of the fluid reservoirs 112, and deliver the fluid 116 to one or more of the fluid dispensers 176. The one or more sections 188 may be coupled together via threading, press fit/friction fit, latches, snap-fit, or other suitable coupling means. In some embodiments, the sections 188 may not be separable and are instead integrally formed.

The fluid passthrough device 100 that is composed of the sections 188, may allow users to switch out the one or more sections 188, or increase/decrease the number of the one or more sections 188 based on the number of different fluids required for a specific procedure. Furthermore, the sections 188 configured to transport gas fluids may be sterilized/cleaned using a different procedure than the sections 188 that transport liquid fluids. It is therefore advantageous that the sections 188 may be easily removed and individually sterilized, because the sections 188 may be more efficiently cleaned separated from the fluid passthrough device 100. It is further advantageous that the section 188 may be split into multiple sub-sections, as each sub-section may be sterilized using a different process.

In some embodiments, each component of the fluid passthrough device 100 may be removable. Namely, the receiving interfaces 122, the valves 164, the actuators 168, the tubes 136, the fluid dispensers 176, and the dispensing interfaces 126 may be separately removable.

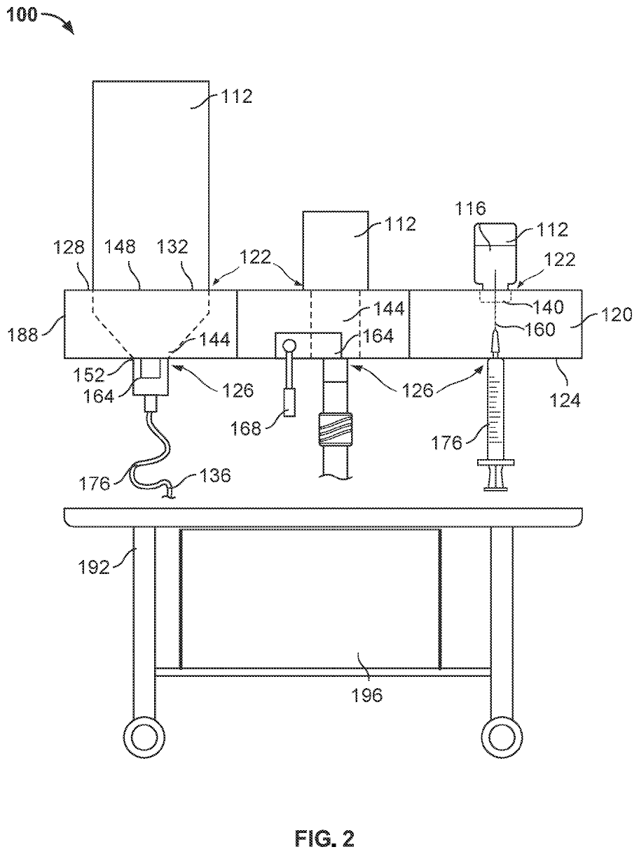

Referring to FIG. 2, the fluid passthrough device 100 is illustrated as mounted on top of a cart 192. In some embodiments, the fluid passthrough device 100 may be transported via the cart 192 to allow for increased maneuverability. The cart 192 may be handled by non-sterile staff in the non-sterile field 108. The cart 192 may house the one or more fluid reservoirs 112 that will be coupled to the fluid passthrough device 100 throughout the procedure. In some embodiments, the cart 192 may include a sterile box 196, where the fluid reservoirs 112 may be stored until the fluid reservoirs 112 are coupled to the fluid passthrough device 100. In some embodiments, the fluid reservoirs 112 may remain within the sterile box 196, while being fluidly connected to the fluid passthrough device 100 (e.g., via the tube 136). Though the cart 192 is shown as having wheels, the cart may be maneuverable via any mechanism, (e.g., treads, rails, etc.).

In some embodiments, the fluid passthrough device 100 may be placed anywhere in the operating room, in any orientation. In some embodiments, the fluid passthrough device 100 may be placed on a table. In some embodiments, the fluid passthrough device 100 may be hung from the ceiling. In some embodiments, the fluid passthrough device 100 may be mounted on a sidewall. In some embodiments, the fluid passthrough device 100 may be mounted on another piece of equipment within the operating room (e.g., a surgical bed, an IV pole, or other equipment). In some embodiments, the fluid passthrough device 100 may be held by a non-sterile personnel during at least a portion of the surgical procedure. In some embodiments, the operating room may include more than one of the fluid passthrough devices 100.

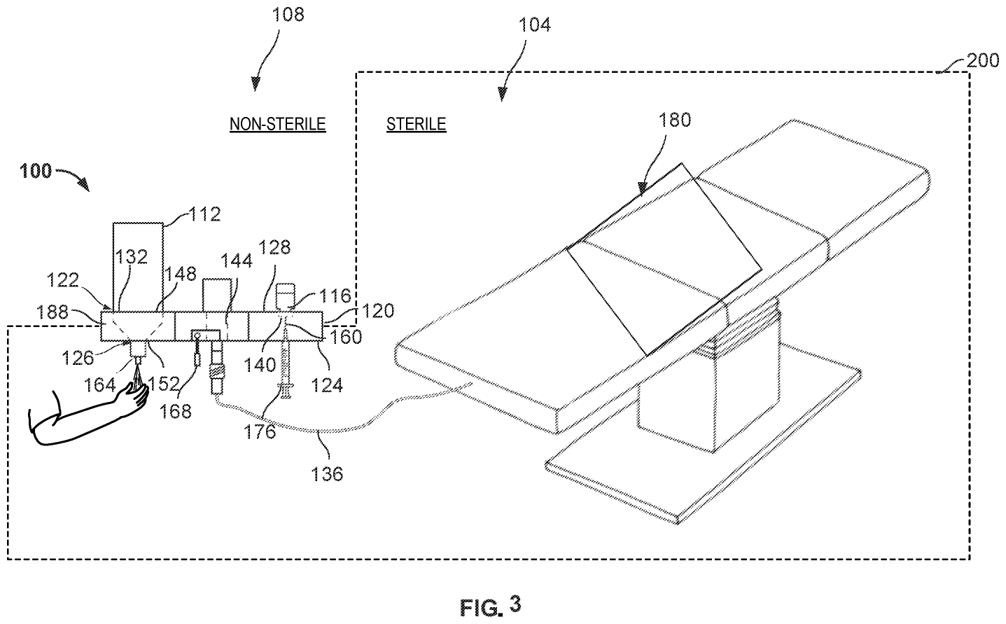

Referring to FIG. 3, an example illustration of the fluid passthrough device 100 within an operating room is shown. The fluid passthrough device 100 is illustrated as being disposed near a border 200 between the sterile field 104 and the non-sterile field 108. As described above, the second surface 128 of the housing 120 of the fluid passthrough device 100 may face the non-sterile field 108, while the first surface 124 of the housing 120 may face the sterile field 104. In some embodiments, the fluid passthrough device 100 is disposed directly on the border 200 of the sterile field 104 and the non-sterile field 108. In some embodiments, the fluid passthrough device 100 is disposed within the sterile field 104. In some embodiments, the fluid passthrough device 100 is disposed within the non-sterile field 108.

Still referring to FIG. 3, in some embodiments the fluid dispenser 176 may extend from the fluid passthrough device 100 to a target area 180 within the sterile field 104. In some embodiments, the target area 180 may be within, on, or near a patient within the sterile field 104. In some embodiments, the fluid dispenser 176 may be filled with the fluid 116, and decoupled from the fluid passthrough device 100, to allow the sterile operators increased range of movement while operating. In some embodiments, the sterile operators may manipulate each of the fluid dispensers 176, while the non-sterile operators determine the rate of fluid flow to each of the fluid dispensers 176 using one or more of the actuators 168 to actuate the valve 164 on each of the one or more channels 144. In some embodiments, the sterile operators may manipulate the fluid dispensers 176, while the sterile operators also determine the rate of fluid flow to the fluid dispensers 176 using one or more of the actuators 168 to actuate the valve 164 on each of the one or more channels 144. In some embodiments, the sterile operators and/or the non-sterile operators are able to actuate one or more of the actuators 168 via the remote or processor described above. In some embodiments, the sterile operators and/or the non-sterile may interact with the processor or graphical user interface on the monitor described above to actuate one or more of the actuators 168 to release the fluid. In some embodiments, the fluid dispensers 176 may be connected to a robotic arm, operable via the manipulatable controllers by the non-sterile or sterile staff.

The non-sterile operators may replace the fluid reservoirs 112 when the fluid reservoirs 112 are depleted, allowing for an increased volume of each of the fluids 116 for the sterile operators. The fluid passthrough device 100 may allow for an increase in size of the fluid reservoirs 112, as the fluid reservoirs 112 do not need to be disposed in the limited space of the sterile field 104. The removal of bulky fluid reservoirs from the sterile field 104 may provide sterile operators performing the surgical operation, more space to maneuver and work. Allowing non-sterile operators to control the rate of fluid flow to the sterile field 104 may reduce the number of sterile operators needed within the sterile field 104 providing further space to the sterile operators to maneuver and work. The reduction in the number of sterile operators in the sterile field 104 may further help to mitigate the risk of infection post procedure, as less operators may lead to a smaller chance of introducing bacteria, viruses, and other disease-causing organisms to the sterile field 104.

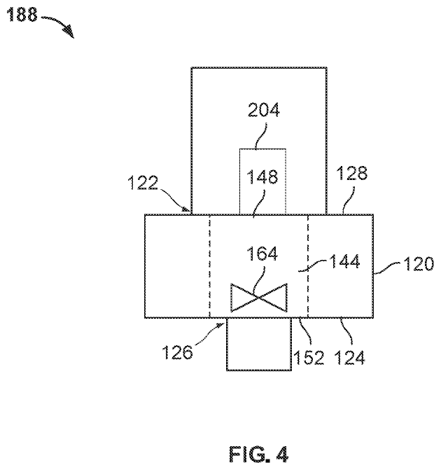

Referring to FIG. 4, an example section of the one or more sections 188 is illustrated. Similar to above the section 188 includes the housing 120, the second surface 128, the first surface 124, the receiving interface 122, the channel 144, the dispensing interface 126, the valve 164, and the actuator 168. The section 188 further includes a prickle 204. The prickle 204 may be disposed in the channel 144 or on the receiving interface 122. As explained above, the reservoir opening 132 of each of the fluid reservoirs 112, may include the seal 140, to ensure that the fluid 116 does not leak from the fluid reservoir 112. In some embodiments, the seal 140 may be broken by the prickle 204, to allow the fluid 116 to flow from the fluid reservoir.

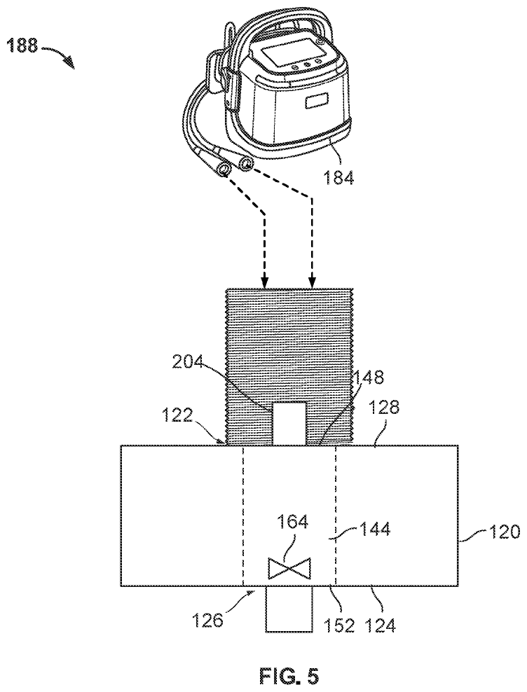

Referring to FIG. 5, an example section of the one or more sections 188 is illustrated. Similar to above the section 188 includes the housing 120, the second surface 128, the first surface 124, the receiving interface 122, the channel 144, the dispensing interface 126, the valve 164, the prickle 204, and the actuator 168. The section 188 is coupled to the pump 184 via the tube 136. The pump 184 may pump the fluid 116 from a reservoir attached to the pump 184, to the section 188 and to the sterile field 104. The pump 184 may be hand-powered, non-hand-powered, or machine powered. The pump 184 may utilize negative pressure via a hand-powered or non-hand-powered mechanism. The pump 184 that is hand powered may be actuated via pushing (e.g., syringe depression), or pumping (e.g., trigger sprayer). The pump 184 that is non-hand-powered implementations may be actuated via pressurized gas (e.g., attachment with pressurized CO2 source readily available in OR), spring-loaded mechanism, pre-pressurized packaging, or a reservoir with a pre-loaded vacuum.

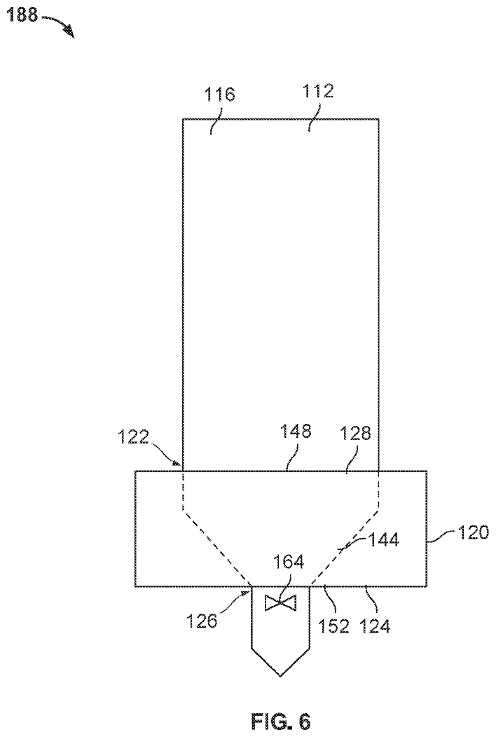

Referring to FIG. 6, an example section of the one or more sections 188 is illustrated. Similar to above, the section 188 includes the housing 120, the second surface 128, the first surface 124, the receiving interface 122, the channel 144, the dispensing interface 126, the valve 164, and the actuator 168. The section 188 illustrates the change in the diameter of the channel 144 between the first opening 148 and the second opening 152. FIG. 6, further illustrates the fluid reservoir 112 coupled to the section 188. The section 188 includes the valve 164 controlled via electronics. In some embodiments, the valve 164 may be controlled both by the actuator 168 that is the electronic signal described above, and by the actuator 168 that is the rotatable knob or lever.

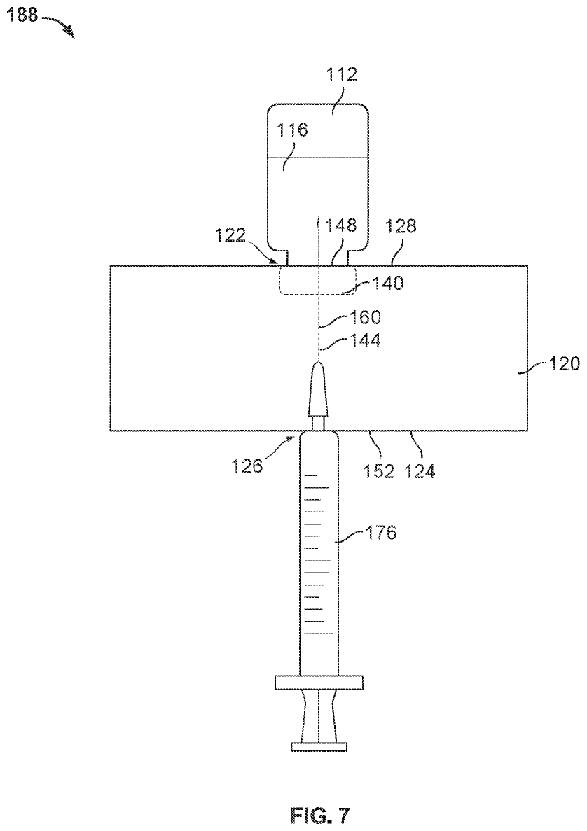

Referring to FIG. 7, an example section of the one or more sections 188 is illustrated. Similar to above the section 188 includes the housing 120, the second surface 128, the first surface 124, the receiving interface 122, the channel 144, and the dispensing interface 126. FIG. 7 depicts the receiving interface 122 that is recessed from the second surface 128 of the housing 120. The fluid reservoir 112 is tipped upside down and is accessed from the sterile field 104 by the cannula 160. The cannula 160 is configured to pierce the seal 140 of the fluid reservoir 112, withdraw the fluid 116, and then be removed from the fluid reservoir 112.

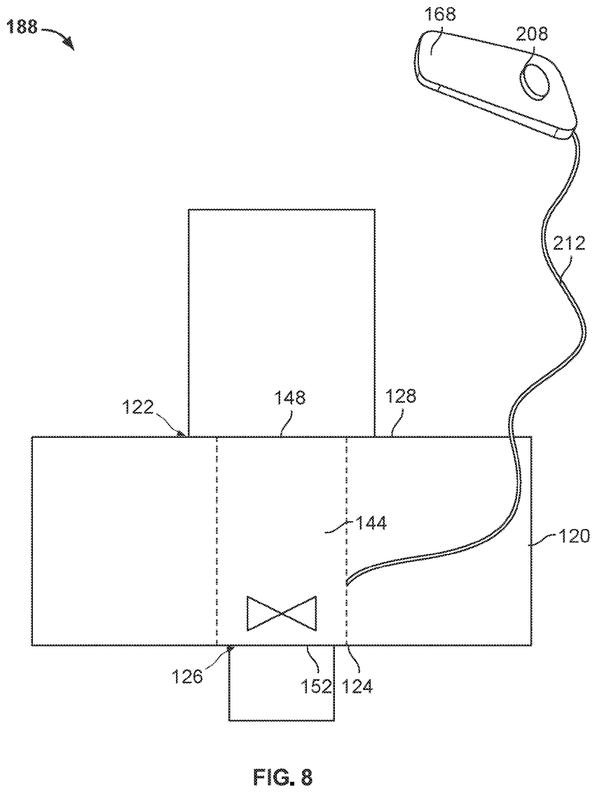

Referring to FIG. 8, an example section of the one or more sections 188 is illustrated. Similar to above the section 188 includes the housing 120, the second surface 128, the first surface 124, the receiving interface 122, the channel 144, the valve 164, and the dispensing interface 126. FIG. 8 depicts the actuator 168 as a remote. The actuator 168 includes a button 208 that may actuate the valve 164 to allow fluid to flow to the sterile field 104. The actuator 168 is depicted as being electrically connected to the valve 164 via a wire 212. In some embodiments, the actuator 168 may be in communication with the valve 164 via Wi-Fi, Bluetooth®, radio waves, or other wireless communication method. In some embodiments, the actuator 168 is controlled by non-sterile operators. In some embodiments, the actuator 168 is controlled by the sterile operators. In some embodiments, the actuator may be handheld. In some embodiments, the actuator 168 may be a foot pedal that allows the sterile operators to dispense the fluid 116 without using their hands.

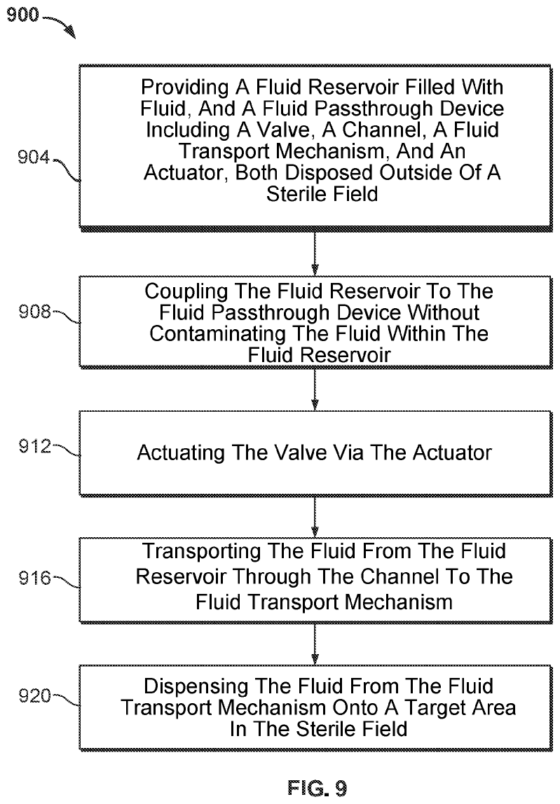

Referring now to FIG. 9, a method 900 is illustrated for utilizing a fluid passthrough device, which may include fewer or more steps than depicted. In some embodiments, the following steps are performed in any order. In some embodiments, two or more of the following steps are performed simultaneously. At a first step 904, the method 900 includes providing a fluid reservoir filled with fluid, and a fluid passthrough device including a valve, a channel, a fluid transport mechanism, and an actuator, both disposed outside of a sterile field. At a second step 908, the method 900 includes coupling the fluid reservoir to the fluid passthrough device without contaminating the fluid within the fluid reservoir. At a third step 912, the method 900 includes actuating the valve via the actuator. At a fourth step 916, the method 900 includes transporting the fluid from the fluid reservoir through the channel to the fluid transport mechanism. At a fifth step 920, the method 900 includes dispensing the fluid from the fluid transport mechanism onto a target area in the sterile field.

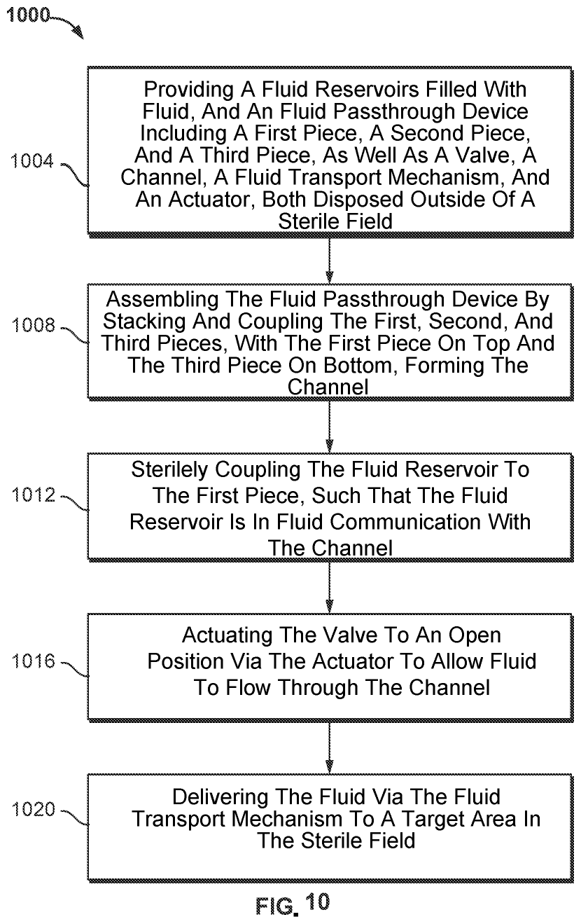

Referring now to FIG. 10, a method 1000 is illustrated for utilizing a fluid passthrough device, which may include fewer or more steps than depicted. In some embodiments, the following steps are performed in any order. In some embodiments, two or more of the following steps are performed simultaneously. At a first step 1004, the method 1000 includes providing a fluid reservoirs filled with fluid, and a fluid passthrough device including a first piece, a second piece, and a third piece, as well as a valve, a channel, a fluid transport mechanism, and an actuator, both disposed outside of a sterile field. At a second step 1008, the method 1000 includes assembling the fluid passthrough device by stacking and coupling the first, second, and third pieces, with the first piece on top and the third piece on bottom, forming the channel. At a third step 1012, the method 1000 includes sterilely coupling the fluid reservoir to the first piece, such that the fluid reservoir is in fluid communication with the channel. At a fourth step 1016, the method includes actuating the valve to an open position via the actuator to allow fluid to flow through the channel. At a fourth step 1020, the method includes delivering the fluid via the fluid transport mechanism to a target area in the sterile field.

As an example application, the systems and methods described in the present disclosure may be used for preparing and laparoscopically delivering a volume of antiseptic solution (e.g., 10-15 cc) as an atomized mist in a short duration of time (e.g., less than one minute), with fewer transfers of antiseptic solution required, thereby reducing the amount of time used to prepare the solution for administration and also reducing the challenge of maintaining the sterile nature of the solution. For instance, the disclosed systems and methods minimize the number of transfers of antiseptic solution in the operating room (“OR”) preparation. As a result of these and other features and advantages, the disclosed systems and methods aim to reduce abscess rates in laparoscopic surgeries.

Although the disclosed fluid passthrough device 100 is described as advantageously spanning a sterile field 104, it is contemplated that the fluid passthrough device 100 is useful as a fluid delivery device for all procedures and hospital settings including for non-sterile procedures, in patient's rooms (both inpatient in the hospital and outpatient at the patient's home or residence), etc.

As described above, the passthrough device 100 may be advantageously used in a patient's room. A patient's room may include one or more sterile fields 104, setup up to place one or more lines at the patient's bedside for delivering antibiotics, saline, or other fluids and gels. One or more of the passthrough devices 100 may be used in each of the one or more sterile fields 104 in the patient's room to organize and provide rapid reloadability of the fluid reservoirs 112 containing the fluid 116 that may be administer to the patient. In some embodiments, the fluid reservoirs 112 may be reloaded while an IV, chest tube, or other fluid dispenser 176 is still connected to the patient, reducing the complexity of fluid delivery to patients.

The disclosed systems and methods enable the delivery of antiseptic solution as an atomized mist during laparoscopic operations, including but not limited to appendectomy, diverticulitis washout, small bowel resection, and colon resection. Advantageously, the disclosed systems and methods remove the need for an external compressor, making delivery of the antiseptic less complicated. Because the disclosed systems and methods are able to deliver antiseptic as a fine atomized mist, a lower total volume of solution is administered, and therefore a lower volume of solution is initially needed.

As still another advantage of the systems and methods described in the present disclosure, time-intensive irrigation is not needed. The disclosed systems and methods instead provide for standardized, easy dispensing of antiseptic through an atomizer.

As noted above, one advantage of the disclosed systems and methods is that the antiseptic solution can be sterilely and easily transferred to the dispensing/atomizing mechanism in a laparoscopic device using the fluid passthrough devices 100 described above. In some particular embodiments, the antiseptic solution can be transferred from outside the surgical field to within the surgical field while maintaining the sterile nature of the antiseptic solution and surgical field, thereby allowing for simpler preparation during an OR procedure. Prefilled antiseptic kitting and/or holistic integration can also be implemented.

In addition to more general transfers of fluid across the barrier between the sterile and non-sterile field, the disclosed systems and methods can be used to prepare and laparoscopically deliver a fine mist of antiseptic solution to an intraabdominal or intrathoracic wound bed at risk for post-operative infection and abscess. One example use case is laparoscopic appendectomy for perforated appendicitis. After removing the appendix, surgeons would benefit from the ability to quickly administer a fine layer of antiseptic solution to the residual inflamed area. This will decrease the rate of postoperative abscess, which is currently 20%.

In general, the systems and methods described in the present disclosure implement the following functions: loading the antiseptic into a reservoir (“L”), such as a fluid reservoir 112; maintaining the sterile field during loading (“M”) 104; dispensing the antiseptic out of the reservoir (“D”), such as via the dispensing interface(s) 126 and/or fluid dispenser(s) 176 of the fluid passthrough device 100; and atomizing the antiseptic laparoscopically (“A”) (e.g., via a trocar).

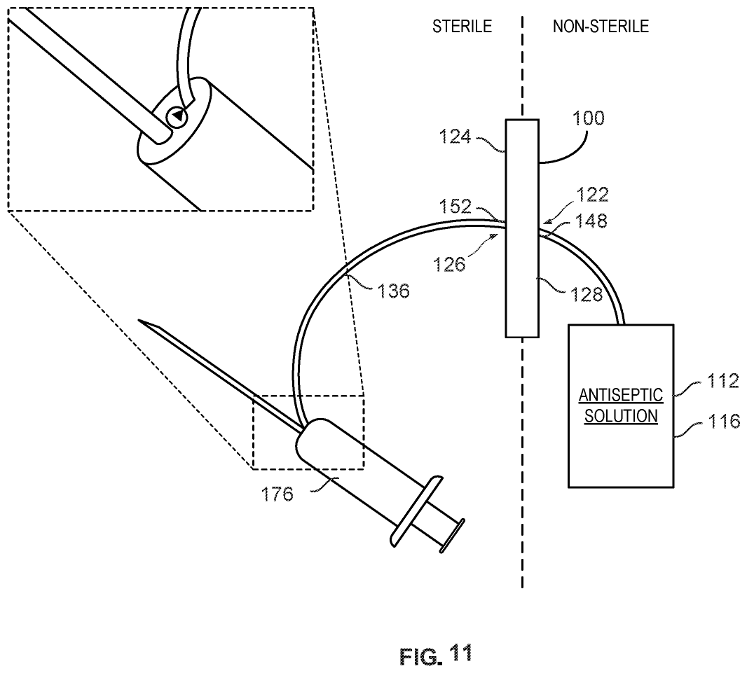

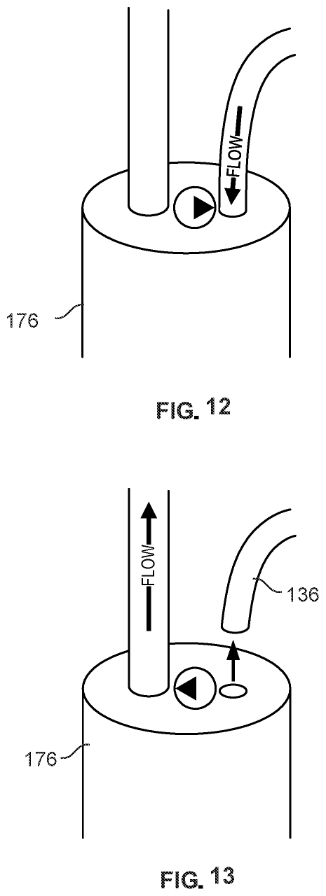

Various embodiments accomplish subsets and combinations of these features. These embodiments will be described below, with L, M, D, and A highlighting the various functions that are accomplished. Examples of laparoscopic devices and loading, maintenance, delivery, and atomization components are shown in FIGS. 11-13.

### EXAMPLE #1

In some embodiments, a fluid reservoir 112, may be sterilely prefilled with a fluid 116, such as antiseptic solution, in a hospital or at a manufacturing facility. The fluid reservoir 112 may be fluidically coupled to a fluid dispenser 176, such as a sterile laparoscopic device (metal or non-metal). In some embodiments, the antiseptic solution is dispensed from the reservoir via a pump 184, such as a hand-powered or non-hand-powered mechanism (D), or other fluid dispenser 176. Hand-powered implementations of the pump 184 and/or fluid dispenser 176 can include: pushing (e.g., syringe depression), or pumping (e.g., trigger sprayer). Non-hand-powered implementations of the pump 184 can include: pressurized gas (e.g., attachment with pressurized CO2 source readily available in OR), spring-loaded, or pre-pressurized packaging. In some examples, the fluid dispenser 176 may be a laparoscopic device that may atomize the fluid 116, i.e., the antiseptic solution, via a nozzle at the patient end of the fluid dispenser 176.

In some embodiments, loading components (L), such as the fluid reservoir 112 and/or fluid 116, and the maintenance (M) components, such as the components of the fluid passthrough device 100 that may be used to maintain the sterility of the fluid 116 (e.g., the receiving interface 122, the channel 144, the dispending interface 126), can be manufactured separately to interface with a commercially available atomizer device. For example, the reservoir 112 (e.g., syringe) can be pre-filled sterilely with antiseptic solution during manufacturing and would interface with a commercially available atomizer (A) on the fluid dispenser 176 via a standard luer-lock connection. The components can be attached together once within the sterile field 104 and the pump 184, fluid dispenser 176, or other dispensing mechanism (D) can be accomplished via the options described above. In some instances, the loading and maintenance components can be distributed as a kit.

### EXAMPLE #2

In some embodiments, a fluid dispenser 176, which may include a sterile laparoscopic device including a dispenser reservoir (metal or non-metal) or a sterile container, is sterilely loaded with fluid 116, such as antiseptic solution or other fluid solution, from a fluid reservoir 112, such as a non-sterile container (i.e., a container whose exterior surface may be non-sterile, but whose contents are sterile), while the sterility of the operating room is maintained. For instance, the fluid 116 can be transferred to a fluid dispenser 176 within the surgical field from a fluid reservoir 112 located outside of the surgical field using the fluid passthrough device 100 described above. The fluid reservoir 112 can be controlled by a non-sterile operator and the laparoscopic device, the fluid dispenser 176, can be controlled by a sterile operator. An extension, such as a tube 136, extending from the fluid dispenser 176 can be utilized to interface with the non-sterile fluid reservoir 112 in order to load (L) the fluid 116 fluid dispenser 176. This extension, tube 136, can be opened in the operative field and begin as completely sterile. Subsequently, there can be two interfaces of the tube 136: a sterile side that remains in the surgical technician's hands, and an environmental side that can interface with non-sterile fluid reservoir 112 and subsequently be detached from the passthrough device 100.

As an example, the passthrough device 100 can be first held in the sterile field 104 and a section of the tube 136 can be placed into a fluid reservoir 112 of antiseptic that is otherwise unsterile. Then, the loading mechanism (L), or pump 184, can utilize negative pressure via a hand-powered or non-hand-powered mechanism. As a non-limiting example, the pump 184 can include pulling or pumping. As another non-limiting example, the pump 184 can include a pre-loaded spring, a gas siphon via pressurized gas flow (e.g., CO2 source readily available in OR), or a reservoir with a pre-loaded vacuum. The pump 184 may be fluidically coupled to the fluid passthrough device 100 to receive fluid 116 from a fluid reservoir 112. Additionally or alternatively, the pump 184 may be a part of the fluid passthrough device 100, such as a dispensing interface 126 and/or fluid dispenser 176. A mechanical release button, or other mechanism, can then physically disconnect the tube 136 from the apparatus such that it falls to outside the sterile field (e.g., by disconnecting the tubing from the receiving interface 122 of the fluid passthrough device 100). After the sterile detachment of the tube 136, the antiseptic solution is dispensed (D) from the fluid dispenser 176 via the pump 184, i.e., the hand-powered or non-hand-powered mechanisms (D), such as those described above. In some embodiments, the hand-powered or non-hand-powered mechanism (e.g., pump 184) for the loading (L) and dispensing (D) functions can be identical, but opposite, which can reduce the mechanical complexity of the device. For example, a pump 184 that is a syringe may utilize a plunger for drawing up the antiseptic into the syringe. The plunger can be reversed to dispense the antiseptic by then depressing the plunger on the syringe. A stopcock or other flow-switching mechanism could be utilized to re-direct the flow from the loading the reservoir to dispensing from the reservoir. As described above, atomization can be accomplished via a nozzle (A) at the patient end of the fluid dispenser 176, or using other means for atomization.

As noted above, in some embodiments, the loading (L) and maintenance (M) components can be manufactured separately to interface with a commercially available atomizer device. For example, a fluid dispenser 176 can be loaded with antiseptic solution (L) sterilely (M) in the operating room via the steps and mechanisms described above. The fluid dispenser 176 may include an atomizer (A). The fluid dispenser 176 may otherwise be attached to the atomizer via a luer-lock connection. In some embodiments, the fluid dispenser 176 and the atomizer can be attached together once in the sterile field, and the dispensing of the fluid can be accomplished via the options described above. The two components can be distributed as a kit.

In some instances, the fluid dispenser 176 and the fluid reservoir 112 can be connected by the tube 136, or via the fluid passthrough device 100 as described above. The tube 136 can then be detached from the sterile container (or fluid passthrough device 100) after loading using one or more detachment mechanisms. As one example, the tube 136 may be connected using a friction fit to one of the receiving interfaces 122 or dispensing interfaces 126 of the fluid passthrough device 100. A mechanical ejection of the tube 136 can then be used to detach the tubing from the fluid dispenser after loading of the solution into the fluid dispenser 176. As another example, a quick disconnect coupling can be used to couple the tube 136 to the fluid dispenser 176. In some embodiments, a threaded nut can be used, such that unthreading the nut can detach the tube 136 from the fluid dispenser. In still other embodiments, a prepositioned hand-squeezed crimp and/or cut in the tube 136 can be used to provide detachment of the tubing from the fluid dispenser 176. For instance, having a prepositioned crimp and/or cut in the tube 136 can enable quick detachment of the tube 136 along the crimp and/or cut after loading of the fluid dispenser 176.

### EXAMPLE #3

In some embodiments, a fluid dispenser 176, such as a sterile laparoscopic device (metal or non-metal) that can sterilely load (L) a fluid 116, such as an antiseptic solution, from a fluid reservoir 112 in the sterile field 104 (M) is provided. An extension, such as a tube 136, of the fluid dispenser 176 can be utilized to interface with a fluid reservoir 112, such as an antiseptic container, in order to load (L) the fluid 116 into the sterile fluid reservoir 112. The fluid dispenser 176 (L) can as an example utilize negative pressure via a pump 184, such as a hand-powered or non-hand-powered mechanism. The pump 184 can include pulling or pumping mechanisms. The pump 184 may also include a pre-loaded spring, a gas siphon via pressurized gas flow (e.g., CO2 source readily available in OR), or a reservoir with a pre-loaded vacuum.

After loading the fluid reservoir 112 (L), the fluid 116 is dispensed (D) from the fluid reservoir 112 via the pump 184 (D) described above. For example, the fluid 116 may be dispensed from the fluid passthrough device 100 as described above. In some embodiments, the pump 184 for the fluid 116 loading (L) and dispensing (D) functions could be identical, but opposite, which can reduce mechanical complexity of the device. For example, a pump 184 that is a syringe may utilize a plunger for drawing up the antiseptic into the syringe. The plunger can be reversed to dispense the antiseptic by then depressing the plunger on the syringe. In some embodiments, multiple channels, or tubes 136, can be used to optimize flow resistance during loading (L) versus dispensing (D). For example, a wider channel can be used to load the reservoir to decrease resistance, while the dispensing and atomization can occur through a higher-resistance channel. A stopcock or other flow-switching mechanism can be utilized to re-direct the flow from the loading the reservoir to dispensing from the reservoir. For instance, negative pressure can be used to draw up solution while positive pressure can be used to expel the solution from the container. As another example, one or more valves 164 can be used to control directionality of the flow. As still another example, an actuator 168, or rotational dial with an aperture that covers an outlet when the inlet is open, and cover the inlet when the outlet is open, can be used to control the directionality of the flow. An actuator 168 that is a thumb switch can alternatively be used to control directionality of flow. As described above, atomization can be accomplished via a nozzle (A) at the patient end of the fluid dispenser 176, or other atomization means.

In some embodiments, the loading (L) and/or maintenance (M) components described above can be manufactured separately to interface with a commercially available atomizer device. For example, a fluid dispenser 176 can be loaded with fluid 116 (L) sterilely (M) in the operating room via the steps and mechanisms described above. The fluid dispenser 176 can then interface with a commercially available atomizer (A) via a standard luer-lock connection or other connection on a commercially available atomizer. The components can be attached together once within the sterile field, and the fluid dispensation (D) can be accomplished via the options describe above. After loading the reservoir (L), the antiseptic solution is dispensed (D) from the reservoir via the hand-powered or non-hand-powered mechanisms (D) described above.

In some embodiments, the pump, or hand-powered/non-hand-powered mechanism for the antiseptic loading (L) and dispensing (D) functions can be identical, but opposite, which can allow for reducing the mechanical complexity of the device. For example, a pump 184 that is a syringe may utilize a plunger for drawing up the antiseptic into the syringe. The plunger can be reversed to dispense the antiseptic by then depressing the plunger on the syringe. As described above, multiple channels, or tubes 136, can be used to optimize flow resistance during loading (L) versus dispensing (D). For example, a wider channel can be used to load the reservoir to decrease resistance, while the dispensing and atomization can occur through a higher-resistance channel. A stopcock or other flow-switching mechanism could be utilized to re-direct the flow from the fluid reservoir 112 to the fluid dispenser 176. For instance, negative pressure can be used to draw up solution while positive pressure can be used to expel the solution from the fluid dispenser 176. As another example, one or more valves 164 can be used to control directionality of the flow. As still another example, an actuator 168 or rotational dial with an aperture that covers an outlet when the inlet is open, and cover the inlet when the outlet is open, can be used to control the directionality of the flow. A thumb switch can alternatively be used to control directionality of flow. The two components can be distributed as a kit.

It will be appreciated that solutions and fluids other than antiseptic solutions can be transferred into the sterile surgical field using the systems and methods described in the present disclosure.

Another embodiment of the device is as described above, with an additional channel that can receive pressurized carbon dioxide from the gas insufflator that is standard in minimally invasive surgeries. The additional channel on the device can have a standard female luer-type connection with a stopcock that can connect with the standard male luer-type connection from carbon dioxide gas insufflators. The gas can mix with the antiseptic solution near the nozzle end of the device to provide an even finer mist than the gasless embodiment.

### EXAMPLE METHOD

A method is also provided for sterilely drawing up a sterile fluid across the sterile field 104 (e.g., a sterile surgical field) and detaching the connection (e.g., a tube 136, a fluid coupling or connection to a container located outside of the sterile field) afterward. As one non-limiting example, the method can include drawing up a sterile antiseptic solution to be delivered to a contaminated surgical (e.g., intraabdominal) wound bed in atomized form.

The method can be accomplished with a laparoscopic atomizer, an antiseptic solution (non-sterile container), and a syringe. The antiseptic solution is transferred onto the sterile field into a sterile container, such as the fluid dispenser 176, and then sterilely drawn up into a syringe. As described above, the fluid dispenser 176 may interface with an external dispense mechanism such as the syringe or an atomizer. The syringe is then connected to the laparoscopic atomizer and depressed in order to dispense the antiseptic solution into the wound bed.

In some embodiments, rather than two separate transfers of antiseptic solution (from container outside sterile field to container within sterile field, then from sterile field container into an atomizer device), the atomizer device may be fluidically coupled to the fluid dispenser 176 that is opened directly onto the sterile field out of its packaging.

In other embodiments, rather than two separate transfers of antiseptic solution (from container outside sterile field to container within sterile field, then from sterile field container into an atomizer device), the fluid dispenser 176 includes an atomizer.

In still other embodiments, rather than two separate transfers of antiseptic solution (from container outside sterile field to container within sterile field, then from sterile field container into an atomizer device), a device that includes an apparatus, such as a pump 184, using negative pressure can be used to sterilely draw up antiseptic directly into an applicator system, or fluid dispenser 176, which in some examples may include using the fluid passthrough device 100 described in the present disclosure to transfer fluid from the non-sterile field 108 to the applicator system in the sterile field 104.

In yet other embodiments, rather than two separate transfers of antiseptic solution (from container outside sterile field to container within sterile field, then from sterile field container into an atomizer device), a device can use negative pressure to directly and sterilely draw up antiseptic, which can then directly interface with the fluid dispenser 176 that includes an atomizer, which in some examples may include using the fluid passthrough device 100 described in the present disclosure to transfer fluid from the non-sterile field 108 to the atomizer in the sterile field 104.

In some embodiments, rather than two separate transfers of antiseptic solution (from container outside sterile field to container within sterile field, then from sterile field container into an atomizer device), a fluid dispenser 176 can be detached after antiseptic is drawn up sterilely across a sterile field 104 using a detachment mechanism, which in some examples may be a part of the fluid passthrough device 100 described in the present disclosure (e.g., a detachment mechanism may be part of the dispensing interfaces 126 of the fluid passthrough device 100). In some embodiments, for example, a device can be used to draw up a medication across a sterile field 104 via the fluid passthrough device 100.

As described above, in some instances, pressurized carbon dioxide, which is readily available in the operating room, can be interfaced with the laparoscopic device and used to deliver a finer mist of antiseptic than what is available to be delivered with currently available atomizers.

It will be appreciated that solutions and fluids other than antiseptic solutions can be transferred into the sterile surgical field using the systems and methods described in the present disclosure. The present disclosure has described one or more preferred embodiments, and it should be appreciated that many equivalents, alternatives, variations, and modifications, aside from those expressly stated, are possible and within the scope of the invention.

1. A device for delivering a fluid from a non-sterile field to a sterile field, comprising:
a housing extending from a first surface facing the sterile field to a second surface facing the non-sterile field;
a receiving interface formed in the second surface of the housing to receive a fluid reservoir containing a fluid;
a dispensing interface formed in the first surface of the housing to receive the fluid from the receiving interface and dispense the fluid into the sterile field.

2. The device of claim 1, comprising a channel fluidically coupling the receiving interface to the dispensing interface.

3. The device of claim 2, comprising a valve to restrict fluid flow through the channel.

4. The device of claim 3, comprising an actuator to actuate the valve between a closed position and an open position.

5. The device of claim 4, wherein the actuator comprises at least one of a knob, a lever, a remote, or a button.

6. The device of claim 1, comprising a fluid dispenser coupled to the dispensing interface to dispense the fluid to a target area in the sterile field.

7. The device of claim 6, wherein the dispensing interface comprises a coupling to fluidically couple the housing to the fluid dispenser.

8. The device of claim 7, wherein the coupling comprises at least one of a threading, a press fit, a friction fit, a Luer taper, a Tuohy Borst adapter, or a needle.

9. The device of claim 6, wherein the fluid dispenser comprises at least one of a spout, gas cannister, fluid bag, syringe, vial, tube, spray nozzle, pump, needle, or atomizer.

10. The device of claim 6, comprising a motion sensor to actuate the fluid dispenser to dispense a volume of fluid when motion is detected by the motion sensor.

11. The device of claim 10, wherein the volume of fluid is a metered volume of fluid.

12. The device of claim 1, wherein the receiving interface comprises a plurality of receiving interfaces fluidically coupled to the dispensing interface.

13. The device of claim 1, wherein the receiving interface comprises a plurality of receiving interfaces and the dispensing interface comprises a plurality of dispensing interfaces fluidically coupled to the plurality of receiving interfaces.

14. The device of claim 1, wherein the housing comprises a solid body.

15. The device of claim 14, comprising a channel formed in the solid body and fluidically coupling the receiving interface to the dispensing interface.

16. The device of claim 1, wherein the housing comprises a hollow body.

17. The device of claim 1, wherein the receiving interface is formed as a recess in the housing.

18. The device of claim 17, wherein the receiving interface comprises a funnel-shaped recess formed in the housing.

19. The device of claim 1, wherein the receiving interface comprises a coupling to fluidically couple the housing to the fluid reservoir.

20. The device of claim 19, wherein the coupling comprises at least one of a threading, a press fit, a friction fit, a Luer taper, a Tuohy Borst adapter, or a needle.

21. The device of claim 1, wherein the housing comprises a plurality of housings, each comprising a different receiving interface formed in the second surface of the housing to receive a fluid reservoir containing a fluid and a different dispensing interface formed in the first surface of the housing to receive the fluid from the receiving interface and dispense the fluid into the sterile field.

22. The device of claim 21, wherein the plurality of housings are removably coupled together.

23. The device of claim 1, comprising:
a channel fluidically coupling the receiving interface to the dispensing interface;
a valve to restrict fluid flow through the channel; and
an actuator to actuate the valve between a closed position and an open position.

24. The device of claim 23, comprising a fluid dispenser coupled to the dispensing interface to dispense the fluid to a target area in the sterile field.

25. A method of sterilely transporting a fluid from a non-sterile field to a sterile field using a fluid passthrough device having a housing extending from a first surface facing the sterile field to a second surface facing the non-sterile field, a receiving interface formed in the second surface of the housing, a dispensing interface formed in the first surface of the housing, a channel fluidically coupling the receiving interface to the dispensing interface, and a valve controlling fluid flow through the channel, the method comprising:
receiving a reservoir containing a fluid with the receiving interface of the fluid passthrough device, such that the reservoir is in fluid communication with the channel and such that the fluid remains sterile;
actuating the valve to an open position;
transporting the fluid from the fluid reservoir through the channel to the dispensing interface; and
dispensing the fluid from the dispensing interface to a target area in the sterile field.

26. The method of claim 25, wherein the fluid passthrough device comprises a motion sensor and wherein actuating the value comprises triggering the motion sensor to actuate the valve to the open position.

27. The method of claim 25, wherein the valve is actuated to a closed position after a metered volume of fluid has been transported through the channel.

28. The method of claim 25, wherein the receiving interface comprises a funnel and the reservoir is received by the receiving interface via pouring the fluid from the reservoir into the funnel.

29. The method of claim 25, wherein the receiving the reservoir with the receiving interface comprises coupling the reservoir to the receiving interface.

30. The method of claim 29, wherein the receiving interface comprises a needle and coupling the reservoir to the receiving interface comprises puncturing a portion of the reservoir with the needle.

31. The method of claim 25, comprising receiving at least one of a tube, a spray nozzle, a pump, a needle, or an atomizer with the dispensing interface, and wherein dispensing the fluid from the dispensing interface comprises dispensing the fluid to the target area in the sterile field via the at least one of the tube, the spray nozzle, the pump, the needle, or the atomizer.

32. The method of claim 25, wherein the reservoir is detached from the receiving interface after the fluid has been transported from the reservoir to the dispensing interface.
